(19) United States
(12) Patent Application Publication
(10) Pub. No.: US 2013/0216522 A1
(43) Pub. Date: Aug. 22, 2013

(54) STABILIZED HUMAN IMMUNOGLOBULIN COMPOSITION

(71) Applicants: Sylvain Huille, Antony (FR); Laetitia Cohen-Tannoudji, Paris (FR)

(72) Inventors: Sylvain Huille, Antony (FR); Laetitia Cohen-Tannoudji, Paris (FR)

(73) Assignee: Laboratoire Francais Du Fractionnement Et Des Biotechnologies, Les Ulis (FR)

(21) Appl. No.: 13/704,375

(22) PCT Filed: Jun. 15, 2011
(86) PCT No.: PCT/FR2011/051358

§ 371 Date: Dec. 14, 2012

(30) Foreign Application Priority Data
Jun. 15, 2010 (FR) 1054721

(51) Int. Cl. A61K 39/395; A61K 47/26

(52) U.S. Cl. CPC A61K 39/39591; A61K 47/26
USPC 424/130.1

(57) ABSTRACT

The invention relates to a liquid pharmaceutical composition comprising human immunoglobulins G (IgGs), comprising at least 200 mM, preferably 250 mM±50 mM, of glycine and between 20 and 100 mg/l of a non-ionic detergent, and having a pH of less than or equal to 4.8.

The invention relates to the formulation of human immunoglobulins G which are useful in therapy.

Many pathologies are currently treated with immunoglobulin G (IgG) compositions. Mention may be made, for example, of primary immune deficiencies with a defect in the production of antibodies, Kawasaki's disease, child and adult immunological thrombocytopenic purpura, secondary immune deficiencies with a defect in the production of antibodies, in particular chronic lymphoid leukemia or myeloma which are associated with recurrent infections, infection of children with HIV associated with bacterial infections, multifocal motor neuropathies, Guillain-Barré syndrome, chronic or severe acute infections with Parvovirus B19, acquired or constitutional immunodeficiency, corticoresistant dermatomyositis, acute myasthenia, idiopathic chronic polyradiculoneuritis, immunological thrombocytopenic purpura, for example associated with HIV infection, stiff-man syndrome, autoimmune neutropenia, resistant autoimmune erythroblastopenia, autoantibody-induced acquired anti-coagulation syndrome, rheumatoid arthritis, and the like.

During the last few years, the very high demand for IgG has created situations of extreme tension over supplies, extending to situations of shortage in Europe and in the United States of America.

In this context, there is an increasing need to produce IgG compositions that can be injected by the intravenous route, from for example human plasma. With the increase in these requirements for IgG, stabilization of these IgG compositions which can be injected by the intravenous route (IgGIV), with a view to their therapeutic use and to their preservation, is critical.

In this regard, it is known that it is necessary to stabilize IgGIVs in order to avoid in particular the formation of aggregates (oligomers and polymers) which are capable of activating the complement system with associated risks of anaphylactic reactions, headaches, fever, blotches, drop in blood pressure (Bolli et al., Biologicals, 2010, 38:150-157). Moreover, the presence of dimers in the IgGIVs has been correlated with drops in blood pressure in vivo. Other physicochemical degradations may also occur during the preservation of IgGs such as, inter alia, oxidation and hydrolysis.

The stabilization of IgGs therefore requires the addition of compounds, which are conventionally chosen from sugars and amino acids, in order to obtain not only non-degraded IgG compositions which are appropriate for therapeutic use, but also IgG compositions with increased stability during storage.

Several formulations of human immunoglobulins intended for intravenous administration have been proposed (cf. in particular patent U.S. Pat. No. 5,945,098, patent applications WO2005/049078 or WO96/07429). A particularly effective formulation for stabilizing immunoglobulin compositions is described in international patent application WO 2004/091656 filed by the applicant. This patent application discloses a composition containing 50 g/l of IgG, 50 g/l of mannitol, 10 g/l of glycine and 50 ppm of detergent, 50 ppm of detergent corresponding to a concentration of 50 mg/l of detergent.

Freeze-dried IgGIV compositions are commercially available, for example under the trade names Polygam™ (American Red Cross), Gammar IV™ (Armour Pharmaceutical Company) and Venoglobulin™ I (Alpha) containing, as stabilizers, 2% glucose, 5% sucrose and 2% D-mannitol respectively.

Liquid compositions of IgGIV which contain, as stabilizers, 10% maltose, 0.16 to 0.24 M of glycine and 5% D-sorbitol are known under the trade names Octagam™, (Octapharma), Gamunex™ 10% (Talecris) and Venoglobulin™ (Alpha), respectively.

However, a need still exists for IgGIV formulations which are well tolerated and which are sufficiently stable for optimum preservation, facilitating their use.

### SUMMARY OF THE INVENTION

The applicant has now developed a pharmaceutical composition comprising human immunoglobulins G formulated with glycine and a non-ionic detergent, at a pH of less than or equal to 4.8.

The inventors have more particularly shown the importance of a low pH for stabilizing this formulation.

A subject of the invention is therefore a pharmaceutical composition comprising human immunoglobulins G (IgGs), comprising at least 200 mM of glycine, preferably 250 mM±50 mM of glycine, and between 20 and 100 mg/l, preferably 35 mg/l±15 mg/l, preferably still 50 mg/l, of a non-ionic detergent, characterized in that the said composition has a pH of less than or equal to 4.8.

Preferably, the composition has a pH of between 4.4 and 4.8. Preferably, the pH is 4.6.

The composition according to the invention is advantageously in liquid form. It may be prepared directly or may be obtained by reconstitution with water, from a freeze-dried product.

Another subject of the invention is a solid composition obtained by desiccation, preferably freeze-drying, of a liquid composition as defined here.

### DETAILED DESCRIPTION

### Definitions

The expression “human immunoglobulins G” or “human IgGs” in the context of the invention is understood to mean polyvalent immunoglobulins which are essentially IgGs, optionally comprising IgMs. They may be whole immunoglobulins, or fragments such as F(ab′)2 or F(ab) or any intermediate fraction obtained in the method of manufacturing polyvalent immunoglobulins.

The term “stability” corresponds to the physical and/or chemical stability of the IgGs. The term “physical stability” refers to the reduction or the absence of formation of insoluble or soluble aggregates of the dimeric, oligomeric or polymeric forms of the immunoglobulins, and the reduction or absence of any structural denaturation of the molecule.

The term “chemical stability” refers to the reduction or absence of any chemical modification of the IgGs during storage, in the solid state or in dissolved form, under accelerated conditions. For example, the phenomena of hydrolysis, deamination and/or oxidation are avoided or delayed. The oxidation of the sulphur-containing amino acids is limited.

### Formulations:

Preferably, the IgG concentration is 100 g/l±20 g/l.

The concentrations are determined in relation to the compositions in liquid form, before desiccation, or after reconstitution in the form of an injectable preparation.

According to a preferred embodiment, the composition contains no mannitol. Indeed, it has been shown that mannitol is not essential for stabilizing the formulation.

More particularly, in a preferred embodiment, the only excipients are glycine and the non-ionic detergent.

An appropriate non-ionic detergent used in the composition according to the invention is advantageously chosen from polysorbate 80 (or Tween®80 which is polyoxyethylenesorbitan monooleate), polysorbate 20 (or Tween®20 which is polyoxyethylenesorbitan monolaurate), Triton® X 100 (octoxinol 10), poloxamers, polyoxyethylene alkyl ethers, ethylene/polypropylene block copolymers and Pluronic®F68 (polyethylene-polypropylene glycol). The non-ionic detergent is preferably chosen from polysorbate 20, polysorbate 80, and/or polyethylene-polypropylene glycol such as Pluronic® F68.

The non-ionic detergents may also be combined with each other.

The compositions of the invention may also comprise other additives. Such an additive may represent a compound chosen from the various categories of stabilizers conventionally used in the technical field of the invention, such as surfactants, sugars and amino acids, as well as an excipient added to the formulation in order to adjust, for example, the pH, the ionic strength and the like, thereof. Alternatively, the composition according to the invention does not comprise other excipients apart from the said glycine and non-ionic detergent. Such a composition has the advantage of offering good stabilization of the immunoglobulin compositions and a reduction in the lengths and costs of preparation on an industrial scale by virtue of the presence of an effective minimum number of excipients as well as the presence of an effective minimum quantity of excipients.

A preferred composition according to the invention comprises:
100 g/l of IgG
250 mM of glycine
50 mg/l of polysorbate 80.

Another preferred composition according to the invention comprises:
100 g/l of IgG
250 mM of glycine
20 mg/l of polysorbate 20.

The immunoglobulins G are generally obtained by fractionation of human blood plasma, and provided in an aqueous medium. The aqueous medium is composed of water for injection which may contain excipients that are pharmaceutically acceptable and compatible with the IgGs. The IgG compositions may beforehand be subject to specific virus inactivation/elimination steps, such as a detergent solvent treatment, pasteurization and/or nanofiltration. The composition according to the invention comprises IgGs which may be polyclonal or monoclonal. The IgGs may be isolated from human or animal blood or produced by other means, for example by molecular biology techniques, for example in cellular systems that are well known to a person skilled in the art. The composition according to the invention is particularly suitable for highly purified IgGs. Advantageously, the IgGs of the present invention are obtained by fractionation of human plasma. Preferred methods of fractionation of human plasma are described by Cohn et al. (J. Am. Chem. Soc., 68, 459, 1946), Kistler et al. (Vox Sang., 7, 1962, 414-424), Steinbuch et al. (Rev. Franc. Et. Clin. et Biol., XIV, 1054, 1969) and in patent application WO 94/9334, these documents being incorporated by reference in their entirety. A method of preparing an immunoglobulin G composition is also described in patent application WO 02/092632, which is incorporated by reference in its entirety.

The liquid compositions according to the invention may be subjected to desiccation in order to obtain a solid form which can be preserved for longer and is more convenient to transport and commercialize. Desiccation is a process for removing water to an extensive degree. It involves dehydration aimed at removing as much water as possible. This phenomenon may be natural or forced. This desiccation may be carried out with the aid of freeze-drying, spray-drying and cryospray-drying techniques. The preferred mode of production of the solid form of the composition for pharmaceutical use according to the invention is freeze-drying. Freeze-drying methods are well known to a person skilled in the art, see for example [Wang et al., Lyophilization and development of solid protein pharmaceuticals, International Journal of Pharmaceutics, Vol 203, p 1-60, 2000]. Other appropriate methods for reducing the degree of moisture or the water content of the composition may be envisaged. Preferably, the degree of moisture is less than or equal to 3% by weight, preferably less than or equal to 2.5%, preferably less than or equal to 2%, preferably less than or equal to 1.5%.

The composition according to the invention may be advantageously subjected to a method for removing or inactivating infectious agents, for example by dry-heating the freeze-dried product.

The solid composition according to the invention, preferably in freeze-dried form, may be dissolved in water for injection (WFI), in order to obtain a formulation for therapeutic use.

### Routes of Administration:

The composition of the invention is useful in therapy, and in particular in a form that is injectable, preferably by the intravenous route. The composition is then in liquid form.

The following figures and examples illustrate the invention without limiting its scope.

### LEGEND TO THE FIGURES

FIGS. 1A and 1B represent histograms showing the measurements of light scattering in dynamic mode (FIG. 1A) or in static mode (FIG. 1B), on immunoglobulin formulations in glycine, at various pH values.

FIG. 2 is a graph which illustrates the results of monitoring of the measurement of turbidity (OD at 400 nm) of immunoglobulin formulations at various pH values, under thermal stress conditions (57° C.).

FIG. 3 is a graph which illustrates the influence of pH on the turbidity of immunoglobulin formulations, under conditions of rotary stirring stress (inversion of the flasks).

### EXAMPLES

### Example 1

### Study of the Influence of pH on the Behaviour of Immunoglobulins

The influence of the pH was tested on 10% concentrated human immunoglobulin G formulations in a 100 mM glycine buffer, the pH being adjusted under non-denaturing conditions, that is to say by dialysis against a buffer with adjusted pH, allowing the target pH to be obtained. Several pH values are tested: 4.6; 5.2; 5.7; 6.4; 6.8.

The state of aggregation of the immunoglobulins is monitored by a light scattering test (angle of90°), after adjustment of the pH (t=0). After adjustment and without applying stress to the formulations, the solutions show different states of aggregation.

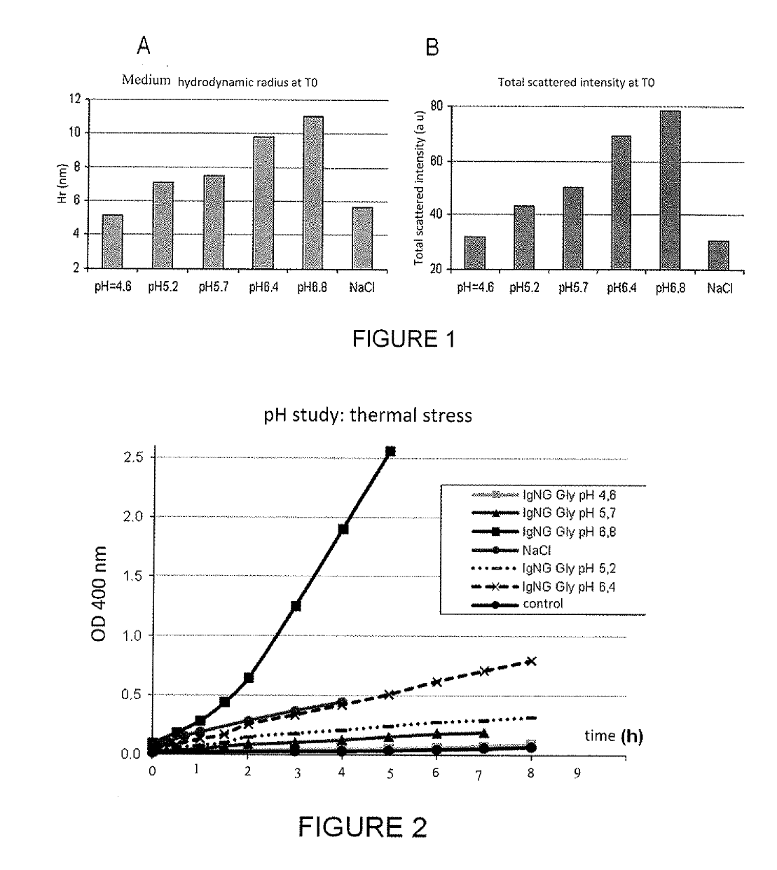

Indeed, the measurements of light scattering in static mode and in dynamic mode show an increase in the submicron aggregation with the rise in the pH (FIGS. 1A and 1B).

The protein solution is composed of various subclasses of immunoglobulins (Ig1, Ig2, Ig3 and Ig4) which exhibit heterogeneity for their isoelectric point (pI), ranging from 5 to 9 approximately. The pH is in fact thought to directly influence the effective charge of the immunoglobulins (Ig), for those which have a low pI (<7.0). In this pH region, some immunoglobulins pass from an overall positive charge to an overall negative charge, which changes the nature of the electrostatic interactions with the other immunoglobulins. The aggregation observed here after adjustment of the pH is similar to oligomerization.

Tests show that in order to have interactions which are repulsive and therefore stabilizing overall, a pH of about 4.6 is favourable.

FIG. 2 illustrates the results of monitoring measurement of turbidity (OD at 400 nm) under thermal stress conditions (57° C.) showing the influence of the pH on the macroscopic aggregation of immunoglobulins. Here again, the pH of 4.6 is the most favourable, the increase in the pH promoting the aggregation phenomena.

A rotary stirring stress (inversion of the flasks) is also performed in order to justify the role of the pH on the aggregation at the water-air interfaces. Measurements of turbidity are carried out after centrifugation and resuspension in order to remove potential microbubbles.

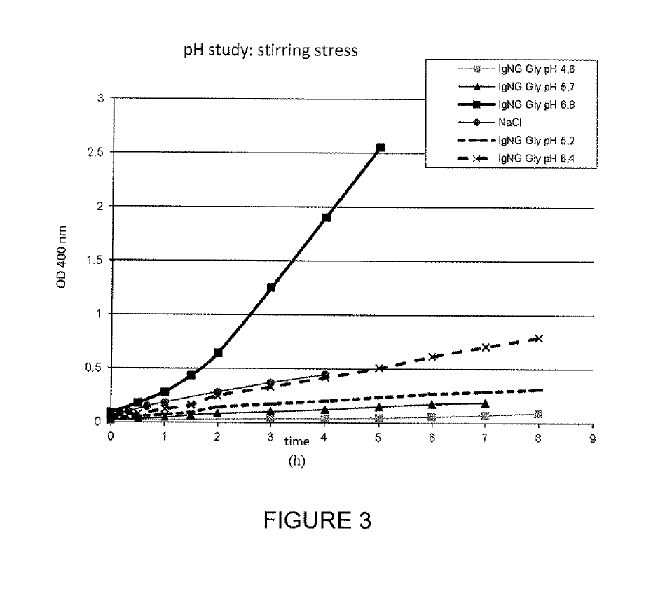

FIG. 3 shows that the aggregation of the immunoglobulins at the interfaces is greatly influenced by the pH.

### Example 2

### Formulations of Immunoglobulins

Several formulations of human immunoglobulins at a concentration of 10% were prepared, with the following excipients, at pH 4.6:

TABLE 1
Formulations tested

| | |
|---|---|
| IgNG | Glycine (93 mM) - Mannitol (175 mM- 32 g/L) - |
| | Tween 80 50 ppm |
| GL | Glycine (200 mM) - Leucine (50 mM) |
| GT20 | Glycine (250 mM) - Tween 20 20 ppm |
| GLT20 | Glycine (200 mM) - Leucine (50 mM) - Tween 20 5 ppm |
| GLM | Glycine (100 mM) - Leucine (50 mM) - Mannitol |
| | (100 mM-18 g/L) |
| GMT20 | Glycine (150 mM) - Mannitol (100 mM- 18 g/L) - |
| | Tween 20 20 ppm |
| GT80 | Glycine (250 mM) - Tween 80 50 ppm |

The immunoglobulins used are obtained from a concentrated solution at 168 g/L at pH=4.7, without any formulation excipient, this solution having been obtained from fractionation of human plasma, and then tangential ultrafiltration. The GT80 and GT20 formulations were prepared by diluting the immunoglobulins in formulation buffers in order to obtain a protein titre of 100 g/l, and the desired concentrations of excipients. Hydrochloric acid was used to adjust the pH.

The formulations were subjected to so-called “accelerated” stability tests by storing them at 25° C. or at 40° C., for 6, 13 or 19 weeks.

The physical degradation was monitored, by analysis of the phenomena of aggregation by HPSEC for monitoring dimers/oligomers and polymers, DLS (dynamic light scattering, measurement of light scattering) for submicron aggregation, counting of subvisible particles (size between 10 and 50 μm), and visual observation (size >50 μm).

The chemical stability was monitored by HPSEC and SDS-PAGE for monitoring fragmentation, and the assay of the anti-HBs antibodies (antibodies to hepatitis B surface antigens) provided an indicator of stability of the Fab function.

The anti-complement activity (ACA) was determined, by measuring the aspecific capture of complement by the immunoglobulins. This test describes the capacity of the immunoglobulins to activate the complement system, it being possible for an excessively high activation of complement to damage the product tolerance during its injection.

The IgNG, GT80 and GT20 formulations gave the best results overall.

The surfactant (Tween) promoted the physical stability of the formulations.

Surprisingly, the presence of mannitol did not prove to be essential, the formulations GT80 and GT20 (free of mannitol) exhibiting stabilities at least as good as the formulation IgNG (with mannitol).

The following formulations are therefore recognized as being the most advantageous:
Formulation GT80: Glycine 250 mM, Tween® 80 50 ppm, pH =4.6
Formulation GT20: Glycine 250 mM, Tween® 20 20 ppm, pH=4.6

### Example 3

### Stability of the Formulation GT80 (pH=4,6)

The stability of the formulation GT80 is compared to that of a formulation IgNG, over 12 months at 25° C. and at 40° C.

TABLE 2
Formulations GT80 and IgNG (10% of human immunoglobulins IgIV)

| | |
|---|---|
| IgNG | Glycine (93 mM) - Mannitol (175 mM- 32 g/L) - |
| | Tween 80 50 ppm |
| GT80 | Glycine (250 mM) - Tween 80 50 ppm |

At T0, the flasks are placed in chambers thermostated at 25° C. and 40° C., and their stability according to the accelerated stability protocol is monitored according to the following schedule:

TABLE 3
Schedule of accelerated stability

| | T0 | T6 W | T13 W | T19 W | T6.5 M | T9 M | T12 M |
|---|---|---|---|---|---|---|---|
| 25° C. | ✓ | ✓ | ✓ | ✓ | ✓ | ✓ | ✓ |
| 40° C. | | ✓ | ✓ | ✓* | | | ✓* |

*only the most distinguishing analyses

After 12 months at 25° C. and 40° C., the formulations GT80 and IgNG exhibit a comparable stability:
The stabilized formulations have identical behaviour from the point of view of chemical degradation: fragmentation is observed at 40° C. by HPSEC and SDS-PAGE and a loss of Fab activity at 40° C.;
The formulations are identical as regards the submicron aggregation observed at 40° C. by DLS and by HPSEC;
A macroscopic aggregation is observed at 40° C. as well as the occurrence of a yellow colour. This colour is identical for the formulations GT80 and IgNG. At 25° C., the 2 formulations are colourless.
The differences in ACA observed remain low. The two formulations change in a comparable manner at 25° C. and 40° C. Mannitol does not contribute to the stability of IgNG in relation to the ACA test.

These results of stability at 12 months confirm that the addition of mannitol has no effect on the stability of IgNG.

Finally, the formulation GT80 consisting of Glycine (250 mM) and Tween 80 (50 ppm) is a stable formulation suitable for commercial therapeutic use. After 12 months at 25° C., all the analyses carried out are in conformity with the European Pharmacopoeia.

1. Liquid pharmaceutical composition comprising human immunoglobulins G (IgGs), comprising at least 200 mM of glycine, and between 20 and 100 mg/l of a non-ionic detergent, wherein the said composition has a pH of less than or equal to 4.8.

2. The composition according to claim 1, said composition having a pH of between 4.4 and 4.8, preferably 4.6.

3. The composition according to claim 1, which composition containing no mannitol.

4. The composition according to claim 1, wherein concentration of immunoglobulins G is 100 g/l±20 g/l.

5. The composition according to claim 1, comprising 35 mg/l±15 mg/l, of non-ionic detergent.

6. The composition according to claim 1, wherein the only excipients are glycine and the non-ionic detergent.

7. The composition according to claim 1, wherein the non-ionic detergent is chosen from polysorbate 20, polysorbate 80 and/or polyethylene-polypropylene glycol such as Pluronic® F68.

8. The composition according to claim 7, comprising:
100 g/l of IgG
250 mM of glycine
50 mg/l of polysorbate 80.

9. The composition according to claim 7, comprising:
100 g/l of IgG
250 mM of glycine
20 mg/l of polysorbate 20.

10. The composition according to claim 1, wherein the immunoglobulins G are obtained by fractionation of human blood plasma.

11. The composition according to claim 1, which is obtained by reconstitution with water, from a freeze-dried product.

12. A solid composition obtained by desiccation, of a liquid composition according to claim 1.

13. A solid composition according to claim 12, wherein dessication is freeze-drying.

14. The composition of claim 1, comprising at least 250 mM ±50 mM of glycine.

15. The composition according to claim 2, which composition containing no mannitol.

16. The composition according to claim 2, wherein concentration of immunoglobulins G is 100 g/l±20 g/l.

17. The composition according to claim 3, wherein concentration of immunoglobulins G is 100 g/l±20 g/l.

18. The composition according to claim 15, wherein concentration of immunoglobulins G is 100 g/l±20 g/l.

19. The composition according to claim 2, comprising 35 mg/l±15 mg/l of non-ionic detergent.

20. The composition according to claim 3, comprising 35 mg/l±15 mg/l of non-ionic detergent.
